(19) United States
(12) Patent Application Publication
(10) Pub. No.: US 2023/0355856 A1
(43) Pub. Date: Nov. 9, 2023

(54) Medical Pump for Endoscopy

(71) Applicant: W.O.M. World of Medicine GmbH, Berlin (DE)

(72) Inventors: Julia Grundmann, Berlin (DE); Thomas Wustrack, Eggersdorf (DE)

(73) Assignee: W.O.M. World of Medicine GmbH, Berlin (DE)

(21) Appl. No.: 18/028,688

(22) PCT Filed: Sep. 30, 2021
(86) PCT No.: PCT/DE2021/000161

§ 371 Date: Mar. 27, 2023

(30) Foreign Application Priority Data
Sep. 30, 2020 (DE) 102020005990.2

(51) Int. Cl. A61M 1/00; A61M 3/02

(52) U.S. Cl. CPC A61M 1/74; A61M 3/0202; A61M 2230/30

(57) ABSTRACT

The present invention relates to a medical pump for endoscopy having a pressure control unit, wherein the pressure control unit takes into account blood pressure changes of the subject.

### CROSS-REFERENCE TO RELATED APPLICATIONS

This application is a 35 U.S.C. § 371 National Stage filing of International Application No. PCT/DE2021/000161, filed on Sep. 30, 2021, which Patent Application claims priority from German Patent Applications No. 102020005990.2 filed on Sep. 30, 2020, the entire disclosure of which is incorporated herein by reference.

### BACKGROUND OF THE INVENTION

### 1. Field of the Invention

The present invention relates to a medical pump for endoscopy having a pressure control unit, wherein the pressure control unit takes into account blood pressure changes of the subject.

### 2. Background of the Related Art

It is known to perform fluid flushing processes during endoscopic examinations and especially during therapeutic interventions. This involves flushing the body cavity (e.g., the bladder, the uterus, or a joint cavity) with a liquid (e.g., saline solution). For this purpose, an flushing fluid can be pumped from a reservoir into the body cavity via a pump, e.g., a roller wheel pump. In the simplest case, the flushing fluid can flow out of the cavity through an opening. However, suction pumps are usually used to pump the flushing fluid back out. The typical sequence of such minimally invasive procedures involves the use of further medical devices for cutting, ablation, or sclerotherapy, each of which may have its own suction line.

During diagnostic procedures in body cavities (e.g., the bladder, the uterus, or a joint cavity (knee, shoulder hip, etc.)), vascular injury and resulting bleeding into the cavity may occur; during therapeutic procedures, such bleeding is usually unavoidable. Such bleeding is undesirable, however, because the blood impairs visibility within the body cavity. It is known to increase the pressure in the body cavity in such cases until no further blood enters the body cavity. In such manual control, the pressure once set is not usually further reduced. However, during such operations, subjects may experience fluctuations in blood pressure. For example, stress can increase blood pressure; relaxation or antihypertensive drugs can also cause blood pressure to drop significantly. In the event of an increase in blood pressure, bleeding may then occur again, necessitating further readjustment of the pressure. A decrease in pressure, on the other hand, is typically not noticed by the operating personnel, so that no adjustment of the fluid pressure in the body cavity takes place here. Excessive pressure, however, can cause flushing fluid to be forced into open blood vessels or into the tissue (interstitium), which can cause complications post-operatively. It would therefore be desirable to provide a control of such a medical pump, which automatically adjusts the pressure of the fluid in the body cavity to the blood pressure.

It is already known from prior art to determine the blood pressure by means of a classic blood pressure cuff and to use the blood pressure determined in this way to control a pump (see EP 3065617 B1). However, the disadvantage of this is that this type of blood pressure measurement is discontinuous. Due to the fact that such blood pressure cuffs completely cut off the circulation of a body part during blood pressure measurement (typically the arm), measurement pauses must necessarily be observed between two measurements to prevent tissue damage due to insufficient blood flow. The alternatively described invasive blood pressure measurement via a catheter located in a blood vessel has the disadvantage that surgical access is required.

### SUMMARY OF THE DISCLOSURE

The present invention solves the above problem by measuring blood pressure by means of at least one sensor that allows continuous, non-invasive blood pressure monitoring without inhibiting blood flow to body parts and without requiring surgical intervention.

The invention therefore relates to a medical device according to claim 1, namely a medical device for flushing cavities in minimally invasive surgery, comprising
(i) a reservoir (1) for the flushing liquid,
(ii) at least one feed line (2) for feeding flushing liquid into the cavity (10),
(iii) one controlled pump (3) per supply line (2) for supplying liquid to the cavity (10),
(vi) at least one suction line (5),
(v) one controlled vacuum pump (4) per suction line (5),
(vi) a waste container (11) connected to the suction line (5),
(vii) at least one pressure sensor (7) for determining the pressure in the cavity (10),
(viii) a control unit (6) for controlling the pressure in the cavity (10),
(ix) at least one sensor (8) for continuous, non-invasive measurement of the blood pressure of the subject (9) during the procedure,
   characterized by that
the control unit controls the controlled pump (3) and the controlled vacuum pump (4) in such a way that the pressure in the cavity (10) is adapted to variations in the blood pressure of the subject (9).

### BRIEF DESCRIPTION OF THE DRAWINGS

FIG. 1 provides an illustration of a medical pump for endoscopy which has been constructed in accordance with an embodiment of the present invention.

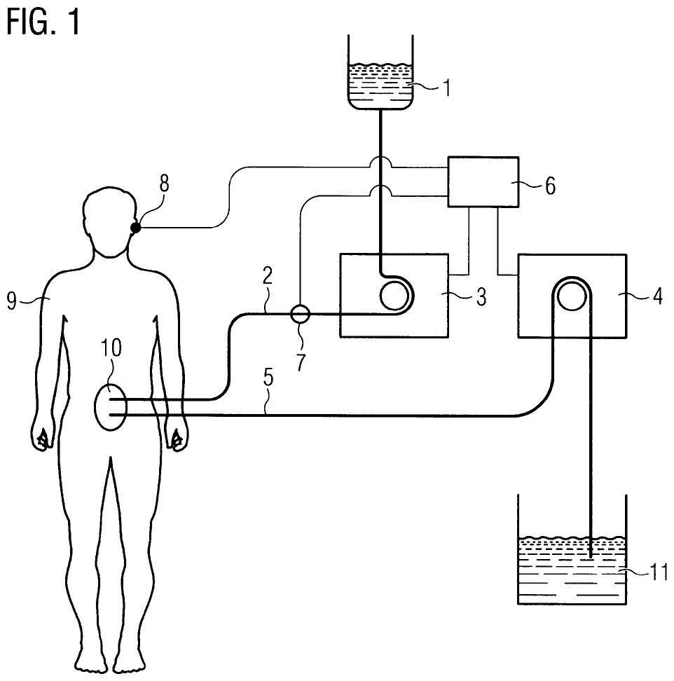

### DETAILED DESCRIPTION OF PREFERRED EMBODIMENTS

Various types of pumps can be used as pumps for liquid supply, which are used in generic devices. Preferred according to the invention is a roller wheel pump, as described in countless variants in prior art. For the device according to the invention, a pump is used which can deliver a fluid flow of 2.5 liters per minute and build up a pressure of 300 mm Hg. Alternatively, another positive displacement pump, such as a diaphragm pump or an impeller pump, can be used for the purpose of fluid delivery, provided they can establish the same fluid flow and pressure.

Roller wheel pumps, alternatively Venturi pumps or diaphragm pumps can also be used for the suction devices. Other types of pumps can also be used. The pump used for suction must be able to generate a vacuum of 450 mm Hg and a liquid flow of 1.5 liters per minute. Roller impeller and diaphragm pumps have the advantage of being very precisely controllable. In comparison, Venturi pumps are less precisely controllable. Improvements in controllability can be achieved via valve control.

A particular embodiment of the invention has at least one roller wheel pump that squeezes a tube in the sense of a peristaltic pump and at least one roller wheel pump for generating a negative pressure for pumping fluid out of the body cavity. Both roller wheel pumps have a sensor for the number of revolutions so that the fluid flow can be precisely controlled. The pumping device has a pressure sensor for determining the pressure in the body cavity. This may be a sensor directly on or in the body cavity. Alternatively, the sensor may be positioned in or on the delivery line or in or on the suction line. It is important to measure the pressure as precisely as possible, which can be determined, for example, using mathematical state spaces (e.g., using Kalman or Luenberger filters).

Essential for the present use is the determination of the subject's blood pressure in a continuous manner. Continuous in the sense of the present invention means that, if possible, every pulse is evaluated. The measurement of the blood pressure is made, therefore, if possible, with each pulse, but at least several times per minute, e.g., five, ten, fifteen or twenty times per minute.

In a first embodiment of the invention, a pulse oximeter may be used for this purpose. Such sensors are described in the prior art. They are based on measuring oxygen-saturated hemoglobin by optical transmission. Such sensors typically consist of a bracket having one or more LEDs on one side, and a photosensitive element on the other side. Such pulse oximeters are typically used to measure oxygen saturation in subjects, with the aforementioned bracket attached to a finger or earlobe. The pulsating blood flow produces a pulsating trace from which blood pressure changes can be obtained. The blood pressure is determined by attenuation with the pulse, which is larger or smaller depending on the expansion of the vessels. This expansion of the vessels is coupled to the blood pressure via the vascular elasticity, whereby the vascular elasticity for the measurement period is to be regarded as static. For the evaluation of the oxygen saturation, measurements are made with more than one wavelength (not further described here) and the course of the pulse attenuation curves is normalized to a mean value. For the evaluation method according to the invention, this averaging or normalization is omitted and the measured raw data is evaluated. The evaluation can be performed in such a way that a baseline of the pulse curve is determined (e.g., using the averaged curve) and its change controls a change in the cavity pressure. Also conceivable are other mathematical methods according to the state of the art for determining the changes in the course of the curve coupled to the blood pressure, such as changes in the mean values for each pulse wave, the minimum-to-minimum changes, maximum-to-maximum changes, courses of the half-amplitude value (value at [(Min+Max)/2]) on rising and falling flank), evaluation of areas under curve sections, evaluation of steepness of curve sections, evaluation of period lengths of curve sections, evaluation of steepness of curve sections, etc. Details of the measurement methodology and evaluation are described in Elter, Peter, Dissertation, U Karlsruhe, Germany, 2001.

Alternatively, blood pressure can be measured using an impedance sensor. This sensor essentially consists of two electrodes that are fixed to the skin (e.g., finger, arm, earlobe). The electrical impedance, i.e. the resistance to a high-frequency alternating current, is measured. Here, too, the pulsations of the blood—and the associated change in the tissue between the electrodes—are determined by changes in the impedance. The fluctuations in impedance can be used to calculate blood pressure in a similar way to the evaluation of the raw data from pulse oximetry. Details of the measurement methodology and evaluation are described in Elter, Peter, Dissertation, U Karlsruhe, Germany, 2001.

It is important to know that the local blood pressure in the small vessels (e.g., in the walls of the uterus or bladder) does not have to be the same as the arterial blood pressure as usually measured on the arm at heart level (e.g., using a blood pressure cuff). Typically, however, local blood pressure in the small vessels is subject to the same (relative) variations as blood pressure in the larger arteries. For example, if the blood pressure in the large artery of the upper arm (brachial artery) drops by 10%, then the local blood pressure in the surgical area typically drops by 10% as well. The sensor used for measurement by pulse oximetry or impedance therefore does not necessarily have to be calibrated at the start of the procedure.

The decisive factor is rather that the pulse oximetry and impedance measurements are performed continuously and the (relative) changes in the pulse oximetry and impedance measurements are used to adjust the pressure in the body cavity. To stay with the above example: If the measured blood pressure drops by 10%, the pressure in the body cavity is also reduced by approx. 10%.

For use in uroscopy and hysteroscopy, it has been found that an adjustment of the fluid pressure in the body cavity from 5 to 20% above the blood pressure produces optimal results. Preferably, the pressure is set about 10% higher than the blood pressure.

### List of Reference Numbers:

(1) reservoir for flushing liquid

(2) feed line for feeding flushing liquid into the cavity

(3) controlled pump for feeding flushing liquid into the cavity

(4) controlled vacuum pump for discharging flushing liquid from the cavity

(5) suction line for discharging flushing liquid from the cavity

(6) control unit

(7) pressure sensor for determining the pressure in the cavity

(8) blood pressure sensor for continuous blood pressure measurement

(9) subject

(10) cavity (body cavity)

(11) waste container

1. A medical device for flushing cavities in minimal-invasive surgery, comprising:
(i) a reservoir (1) for a flushing liquid,
(ii) at least one supply line (2) for feeding flushing liquid into a cavity (10),
(iii) one controlled pump (3) per supply line (2) for supplying liquid to the cavity (10),
(vi) at least one suction line (5),
(v) one controlled vacuum pump (4) per suction line (5),
(vi) a waste container (11) connected to the at least one suction line (5),
(vii) at least one pressure sensor (7) for determining a pressure in the cavity (10),
(viii) a control unit (6) for controlling the pressure in the cavity (10),
(ix) at least one sensor (8) for continuous, non-invasive measurement of a blood pressure of the subject (9) during the procedure,
wherein
the control unit controls the controlled pump (3) and the controlled vacuum pump (4) in such a way that the pressure in the cavity (10) is adapted to variations in the blood pressure of the subject (9).

2. The medical device according to claim 1, wherein at least one sensor (8) for continuous measurement of the blood pressure of the subject (9) is selected from a pulse oxymeter sensor or an impedance sensor.

3. The medical device according to claim 1, wherein the controlled pump (3) for liquid supply is a roller wheel pump.

4. The medical device according to claim 1, wherein the controlled vacuum pump (4) is a roller wheel pump.
